(19) United States
(12) Patent Application Publication
(10) Pub. No.: US 2005/0053519 A1
(43) Pub. Date: Mar. 10, 2005

(54) Delta cup

(76) Inventor: Jack V. Smith, Arden, NC (US)

Correspondence Address: Jack V. Smith, P.O. Box 156, Arden, NC, 28704, US

(21) Appl. No.: 10/660,085

(22) Filed: Sep. 10, 2003

(51) Int. Cl. G01N 33/00

(52) U.S. Cl. 422/58

(57) ABSTRACT

The present invention is a device for assaying and collection of biological and other specimens and is especially designed for the collection and determination of the presence of chemical constituents in clinical chemistry, pregnancy, drugs of abuse testing, infectious disease testing, and other fields of analysis of fluids.

### BACKGROUND OF THE INVENTION

1. Field of the Invention

The present invention is a method that elates to assaying and collecting biological and other specimens and is especially designed for the collection and determination of the presence of chemical constituents in drugs of abuse, urinalysis, infectious disease, clinical chemistry and other areas of analysis. The present art provides a simple and convenient method for the collection, testing, photocopying, and reading by an instrument or other device of results that the prior art cannot provide.

Some of the collection devices of the prior art for urine for example were not designed to be used for analysis. These devices were strictly designed to collect urine on the ward of a hospital and then sent to the laboratory for testing. Or, the nurse would collect a urine and take it back to the nurse's station and test the urine commonly with a urine dipstick. There are several drawbacks to this. First the nurse will have to have an open urine container at the nurses station. This presents a biological hazard that the nurse, doctors, patients, and passerby's would be exposed to. The chances are spillage of the urine specimen or any liquid for that matter is high when ever you have an open container present. With this specimen now present at the nurses station after collection the pressure to test and dispose of the specimen is increased for workload, safety and storage area (clutter) reasons alone. The next step for the nurse would be to take the urine specimen and dip a dry chemistry test strip into the urine and analyze for urine analytes of interest (constituents). The constituents that are commonly analyzed in urine specimens are glucose, pH, specific gravity, bilirubin, urobilinogen, nitrite, protein, red blood cells (hemoglobin), ketones, white blood cells (human leukocyte esterase), bladder cancer, human chorionic gonadotropin (HCG) and drugs of abuse. Once the test strip has been removed from the specimen it needs to be compared to a color chart to determine the concentration of the urinary constituents. The nurse will then wait the pre-required time to read each and every color pad and or test line as designated by the package insert for the test strip by the manufacturer. After analysis the nurse does not want to lay this strip on the counter for contamination reasons. The nurse may possibly use a paper towel to lay the strip on. Once the results are recorded the nurse will then properly dispose of the test strip and urine specimen and container. Resulting in an inordinate amount of risk, time, and labor.

2. Description of the Related Art

The present is device that is designed to collect and assay the presence of urinary constituents (analytes of interest) in a biological urine specimen. This specimen could come from humans, animals or other sources submitted for analysis of the analyte of interest. That is to say for example that the present device (invention) is designed to be used for the collection and detection of glucose in the urine specimen or the device is used to collect urine and detect virulent disease causing viruses such as HIV, proteins, viruses, drugs of abuse, drug metabolites, clinical analytes of interest, and therapeutic drugs.

There is no prior that produces the unexpected results of the present device and the answers to a solution to that was never before even recognized that the present art provides. The prior art teaches away from the present art in that it goes in a completely different direction. That is to say that the collection devices of the past for urine were not designed to be used for analysis but strictly collection. For example these devices were strictly designed to collect urine on the ward of a hospital and then be sent to the laboratory for testing. The collection device was designed to collect urine and test however these devices are cumbersome, expensive, and not designed for the specific purpose of testing biological constituents. There are some devices that are designed to perform analysis of certain constituents but in a cumbersome and messy manner and these devices were not designed to collect urine for any period time and have numerous drawbacks and limitations when related to the advance that the present device brings to the art.

A thorough search of patents, publications, and research revealed no relative art (i.e., prior art) showing any direct correlation to this novel invention. The search included the USPTO (United States Patent Office) data base with no patents issued for a device designed specifically for biological specimen or other fluid collection and testing that is unique this device. However, the following art will be mentioned to further illustrate the novelty of the present art and the obvious advancement to the current art. The following patents, without exception do not mention the use of a cup for collection and analysis of biological specimens for detecting specific analytes of interest with the additional ability to photocopy each side of the device for recording and/or analysis of the results.

It is known in the art that the urine matrix is very complex and consists of many urinary constituents which create strong buffering and interference problems (e.g. cannibal-like enzymes such as protease) that have to be overcome to provide a method that can be used for the general population with precision and accuracy. Simply because a technique can accommodate a liquid sample does not imply that it can be successfully used with any liquid test matrix. Such successful adaptation of test techniques to accurately deal with specific sample matrices aren't often “obvious” to any scientist. The same can be said of certain types of techniques used to analyze urine. For instance, the art is replete with examples of devices that provide dry chemistry dipsticks for dipping into a urine container and reading the result. However these dipsticks devices have crossover contamination problems from reaction pad to reaction pad because the dipstick is covered with urine and the urine from back and forth from reaction pad to reaction pad. However, the present art will demonstrate in detail the techniques developed that will overcome these type of interferences and issues with the prior art.

The number of collections of biological specimens is very large in the United States and worldwide. The numbers are in the hundreds of thousands of specimens per day collected in urine containers for drugs of abuse screening, adulteration testing, urinalysis, infectious disease testing, clinical chemistry and other testing. Since very large numbers collected are involved it is very important to the art for a device designed to answer the problems of the current art that will be effective, safe, simple, and cost effective. No current device in the art solves these problems until this invention which can provide millions in savings with regards to rising health care cost.

Specimens collected for drugs of abuse testing sometimes require that the specimen integrity and chain of custody be validated. The adulteration of samples submitted for drug testing is unacceptable. The assay(s) run on any specimen submitted for any analysis is only as good as the specimen collected.

Also with the onset of HIV (human immunodeficiency virus), STD's (sexually transmitted diseases), hepatitis and other infectious diseases the health risk associated with the handling of body fluids has increased exponentially over the last few years. Therefore, if a device is invented that can provide added safety it is very likely that it will save lives.

The multiple steps of specimen collection as required with the prior art are hazardous with regards to infectious diseases. First the sample is collected in a container then the specimen is transferred to another container for testing in a device, test tube, or instrument. In the case of drugs of abuse testing the sample has to be split to another container before it is tested so that the original container is not contaminated with the test device (in case of cross contamination from the test device). These multiple steps procedures of potentially infectious material have required the manual use of test tubes, pipettes, syringes, or other devices used in the transfer of specimens from collection device to the final container use for analysis. Then of course after the assay is completed the assay container and/or the specimen has to be discarded.

Another issue with the prior art is the possible misidentification or mislabeling of the specimen collected anytime the specimen has to be removed from the original container. This could in an erroneous result for the original specimen. Imagine a urine submitted for an HIV test and it was mix up with another specimen because of mislabeling and as erroneous result was reported. The implications are grave.

Different attempts at providing an effective collection device have been attempted but all have failed for multiple reasons. U.S. Pat. No. 5,403,551 to Galloway, describes a cup for collecting and analyzing a specimen but this device has multiple drawbacks. The device requires that the user to invert the container prior to analysis. When the container is inverted it leaks quite profusely. Which does not answer the contamination problem. The device is assay part of the device is attached to the collection cup and is not part of the cup and requires a number of chambers, channels, and other means that add to the cost and complexity of the device. In addition, the Galloway device requires the use of a plenum (a space in which a gas, usually air, is contained at a pressure greater than atmospheric pressure. And this device does not have anyway to simply collect and analyze the specimen in a single step while providing a way for the analyst to photocopy and or have the results recorded by an instrument independent of the device thereby removing subjective analysis. In, addition, U.S. Pat. Nos. 5,096,813 and 4,769,215 to Ehrenkranz provides drug testing urine collector type devices that includes perhaps the most complex devices ever designed for urine collection. The complexity of the devices alone would raise the cost of the devices to a level that it would infeasible to market and sell the devices. The devices have almost as many problems as the Galloway device. They actually has adulteration detection reagents in the reservoir. This is a major problem with regard sample contamination. The complexity of manufacturing and the contamination issues from the adulteration detection reagents to name a few are major drawbacks to these devices. And this device does not have anyway to simply collect and analyze the specimen in a single step while providing a way for the analyst to photocopy and or have the results recorded by an instrument independent of the device thereby removing subjective analysis.U.S. Pat. No. 5,096,813 to Krumhar is a device designed specifically to for storage and the detection of oxygen and has no relative bearing on the present invention. It is however, a device used for storage and by no means can be compared to the present device which can analyze a specimen at the point of collection, without tilting the cup, or pouring into another device, etc. And this device does not have anyway to simply collect and analyze the specimen in a single step while providing a way for the analyst to photocopy and or have the results recorded by an instrument independent of the device thereby removing subjective analysis.

Other patents such as the following have no relative bearing from the present art because there is no semblance in shape of function they are mentioned just to further illustrate the absolute unique properties of the present art.

For instance, U.S. Pat. No. 2,953,132 discloses a solution bottle with an inwardly projecting tube and a rubber stopper and an associated dispenser bottle, which is adapted to introduce the medication into the solution bottle. And this device does not have anyway to simply collect and analyze the specimen in a single step while providing a way for the analyst to photocopy and or have the results recorded by an instrument independent of the device thereby removing subjective analysis. U.S. Pat. No. 3,066,671 discloses a disposable additive container provided with a cover formed with a shaft-guiding sleeve. The shaft-guiding sleeve receives an infusion holder and an additive container. And this device does not have anyway to simply collect and analyze the specimen in a single step while providing a way for the analyst to photocopy and or have the results recorded by an instrument independent of the device thereby removing subjective analysis.

Another patent, U.S. Pat. No. 3,608,550 discloses a transfer needle assembly for transferring fluid from a fluid source to a fluid collection container. The needle assembly includes a first cannula mounted on a support means, which engages the collection container and is adapted to be connected at its forward end to the fluid source and at its rear end to the collection container. A second cannula is mounted on the support means and is adapted to be connected at its forward end to the fluid source and at its rear end to the atmosphere allowing fluid to be transferred from a fluid source to a collection container by atmospheric pressure when the volume within the collection container is sufficiently increased. And this device does not have anyway to simply collect and analyze the specimen in a single step while providing a way for the analyst to photocopy and or have the results recorded by an instrument independent of the device thereby removing subjective analysis.

Another patent, U.S. Pat. No. 3,904,482 discloses an apparatus and method for the collection, cultivation and identification of microorganisms obtained from body fluids. The apparatus includes an evacuated tube containing a culture medium, an inert gaseous atmosphere and a vent-cap assembly. The tube containing the culture medium is fitted with a stopper for introduction of body fluid by means of a cannula and, after growth of the organisms, transfer of the cultured medium is completed for subculturing or identification procedures. And this device does not have anyway to simply collect and analyze the specimen in a single step while providing a way for the analyst to photocopy and or have the results recorded by an instrument independent of the device thereby removing subjective analysis.

Another patent, U.S. Pat. No. 4,024,857 discloses a micro device for collecting blood from an individual or other blood source into a blood sampler cup. The cup has a removable vented truncated cone shaped top with a capillary tube passing through a well formed in the top proximate to the inside wall of the cup to deliver blood directly from the blood source to the cup. And this device does not have anyway to simply collect and analyze the specimen in a single step while providing a way for the analyst to photocopy and or have the results recorded by an instrument independent of the device thereby removing subjective analysis.

Another patent, U.S. Pat. No. 4,116,066 discloses a device for the collection of a liquid, such as urine comprising an open-ended urine collection container provided with a hollow cannula attached to its bottom. The cannula is slotted near its base, and serves as the conduit through which liquid may be transferred from the container to an evacuated tube. When the stopper of the evacuated tube is punctured by the cannula, the pressure difference causes liquid deposited in the container to be drawn through the slot into the hollow cannula and into the tube. And this device does not have anyway to simply collect and analyze the specimen in a single step while providing a way for the analyst to photocopy and or have the results recorded by an instrument independent of the device thereby removing subjective analysis.

And yet another attempt to solve this problem is seen in U.S. Pat. No. 4,300,404, in which a container is developed having a liquid container with a snap fit lid. The lid is provided with a cannula which extends into the lower end of the container and which projects through the lid at its upper end so as to be able to pierce the stopper of an air-evacuated tubular container. The container is also provided with a depressed bottom to assure the maximum collection of fluids and the lid is provided with a recess to accommodate the air-evacuated tube. And this device does not have anyway to simply collect and analyze the specimen in a single step while providing a way for the analyst to photocopy and or have the results recorded by an instrument independent of the device thereby removing subjective analysis.

None of the afore mentioned devices teach, illustrate or have anything in common with the present art, in fact all of the afore mentioned devices could be combined and still not produce the present device. Therefore, there is no chance that these devices could used as prior teachings of the present art.

While the prior art provides certain devices for the collection of fluids or other types of samples the prior art however suffers from a certain number of drawbacks.

The inflation, insertion, and closure of the prior art devices all require multiple steps and are not simple efficient method to collect and analyzed urine without the risk or contamination, spillage, or other problems. All of the prior art requires tedious and complex methods for use. For instance, the one prior requires that certain the cup be tilted prior to analysis increasing the risk of leakage and contamination as the specimen leaks out of the container. Another device requires the use of a plunger (syringe) for use and yet another requires the use of tilting and a plenum. These are just some, not all, of the limitations of the prior art.

The present device is designed for the analysis of biological specimens on site. That is to say the device can be used for the collection and analysis of the specimen within the container without removal of the specimen and without have to adjust the lid of the container, use a plunger, a plenum, or other multiple steps as required by the prior art. The specimen can be analyzed immediately at the point of collection or sent to the lab and tested the next day. The device can be used for long storage of a specimen before testing and/or for immediate analysis. This removes the risk of contamination, mislabeling, chain of custody, and cross over contamination and offers the added ability to copy results from any side of the collection device for recording of test results or the device can be easily read by an instrument providing a means of removing subjective analysis that is inherent the novel and inventive design of the present device.

### SUMMARY OF THE INVENTION

The present invention is designed to advance the art of urine collection and on site (at the point of collection) analysis past the prior arts drawbacks and provide a collection and analyzing cup that is simple to use, requires minimal instruction, has the minimum number of parts, and is cost effective. Another object of the present invention is to provide a method that allows for an easily automated process and readable. This is to say that the device is designed to be copied from any of the three sides that the test device can be read from.

Correspondingly, another advantage of the present art is to provide a collection and analyzing device that will allow the user to collect the specimen in the cup, place the lid on the cup to prevent any biohazard accidents or contamination, analyze the specimen without having to further manipulate the cup like tilting, using a plunger, a plenum, etc. This is truly a one step process which is not currently known in the art.

It has been found that the foregoing objects of the present art are accomplished in accordance with this invention by providing a collection and analyzing cup that is designed to collect the specimen and immediately have the lid secured onto the top of the cup. The cup is designed for long term storage if necessary before and after analysis.

The present invention provides a method of specimen collection and analysis as defined above, and the method being characterized by the following steps:
a) collecting the specimen in the cup;
b) placing the lid on the cup and closing;
c) and recording the results of the analysis without the use of a plunger or the requirement of tilting the specimen by direct observation or photocopying the results.

Other aspects and advantages of the present invention appear more clearly from reading the following detailed description of the preferred embodiment of the invention, given by way of example and made with reference to the accompanying drawings. Such as the determination of exactly how the device works. A thorough search of the literature reveals no relative art resembling this technology; therefore, this invention is clearly a novel in creation, and is not obvious to anyone skilled in the art, in fact the prior art devices teaches away from the present art in that the prior art requires that the cup be tilted in order for the device to be used (this is not a requirement of the present device in fact the present device is a teaching of simplicity with no manipulation of the cup as a requirement) and the prior art devices teach away from the present device in that some prior art require that the lid be off the container to activate, and the prior art teaches away from the present device in that the prior teaches the use and requirement of a plenum which is a pressurized space, etc. The present device teaches the use of a chamber that does not require pressure and a stable pressure to a vacuum would actually be preferred. There are certain aspects of the present art that can be found in the prior art (e.g. the use of a cup) but no prior has advanced the art of specimen collection and analysis as much as the present art. This art solves an unrecognized problem that was never before even recognized. Specifically this allows for the user the unexpected results of using a device that is simple, efficient and cost effective that only utilizes a cup and activation device without the use of plungers, plenums, tilting, etc., for a much more effective and safe method of collection and analysis of a specimen which can be read and photocopied and analyzed by instruments which is inherently made possible by the novel design of the device.

The collection and assaying device, in accordance with the present device, for both collecting and analyzing specimens, includes a container (cup) having an opening for collection of the specimen and a chamber for storing the collected specimen. A cap provides a means for sealing the container opening and an assay means which, is not attached but integrated into the container providing for chemically analyzing the specimen. In addition, after a sample has been introduced into the device the results can be recorded by a photocopier or other means made possible by the unique design of the device.

The specimen can be a biological sample (urine, etc.) or other type of fluid.

It important that the means are provided for introducing a portion of the collected specimen within the chamber into the assay means when the cap is not on the container. However, this device and testing means does not require that the cap be in place. By placing the cap into position there is no requirement for removing the sample from the assaying device in order to conduct chemical analysis.

Therefore, the apparatus of the present device (invention) totally removes the need to transfer the collected sample from the device in order to conduct a chemical analysis as is the case with prior art devices. As mentioned this has a significant importance with regard to safety, biohazard, time, accuracy, ease of use and savings.

Additionally, one embodiment of the present device is particularly suitable for Infectious disease, Drugs of Abuse, Pregnancy, Urinalysis Testing of biological fluids which includes a convenient method for the photocopying of result. And, since the fluid specimen never leaves the device, if a positive test for HIV (infectious disease), or drug, etc., is indicated, the entire device may be removed or shipped to the laboratory or other facility for further or confirmation testing.

Additionally, the present device, the assay means may include chromatograph, thin layer chromatography and dry chemistry hybrid, dry chemistry test pads attached to lateral flow device or other material assay means integrated in the container for enabling direct visual observation of the assay results. Therefore, no additional steps are necessary for effecting an analysis of a biological specimen.

As mentioned above, the assaying means of the device in accordance with the present invention includes a means for preventing biological fluid from entering the assay means during the collection of the body as is the case with all prior art (as discussed with the plenum and the tilting as required by one particular art (note: this could happen with the prior art during collection the cup could be tilted while the specimen is entering the cup and the specimen goes directly into the plenum).

A wicking means may be provided but is not required for enabling the biological fluid to enter the assaying means or aid the assaying means in the movement of fluid from one end of the assaying means to the other.

The assaying means is integrated into the container sidewall (not bottom or top), and no activation means is required by the present art and is a limitation of the prior art.

The assaying means may contain a plurality of separated thin layer chromatograph strips with each strip comprising means for chemically analyzing the biological fluid for a different analyte, or chromatograph, or thin layer chromatography and dry chemistry hybrid, or dry chemistry test pads attached to lateral flow device or other material assay means.

And the assaying means may include a wick for evenly distributing the biological fluid to each of the assay means if more than one is present. The wick can be at both ends of the assaying means or at just one end of the assay means, or not present at all.

### BRIEF DESCRIPTION OF THE DRAWINGS

Other objects, features and advantages of the invention will become obvious from the following detailed description of the invention when taken in conjunction with the accompanying drawings, in which:

FIG. 1 is a plan view of one embodiment of the Delta Cup made in accordance with this invention generally showing the container, activation device, and assaying means;

FIG. 1A-1E are plan view and cross section views of the assay means which are inserted into the slots in the Delta Cup in accordance with this invention generally showing the container, assay device, and assaying means;

FIG. 2 is a cross section view of FIG. 1 prior to placing the lid onto the container generally looking down into the container from the top;

FIG. 3 is a plan view of the Delta Cups lid which can be placed and snapped onto the top of the container;

FIGS. 4 and 4A is a plan view of the Delta Cup with at least one flat side and cross section view of the lid for the single side cup which can be placed and snapped onto the top of the container.

### DETAILED DESCRIPTION OF THE PREFERRED EMBODIMENTS

The present invention will now be described more fully with reference to the accompanying drawings, in which the preferred embodiments of the present art invention are shown. It is understood from the embodiments that a person skilled in the art may make variations and modifications without departing from the spirit and scope of the invention. Such as changing the size or shape of a Delta Cup, the optional addition of a wick, or the addition of a magnifying side wall to allow for easier reading and automation of the assaying mean results, the addition of more than one slot on one side of the Delta Cup. In such that the present arts cup is capable of having as many 15 to 20 slots for analysis of multiple assays simultaneously or the device could have just one slot for analysis on a single wall.

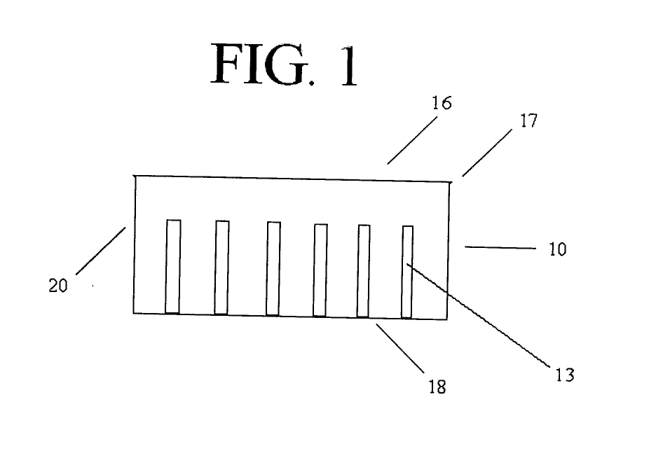

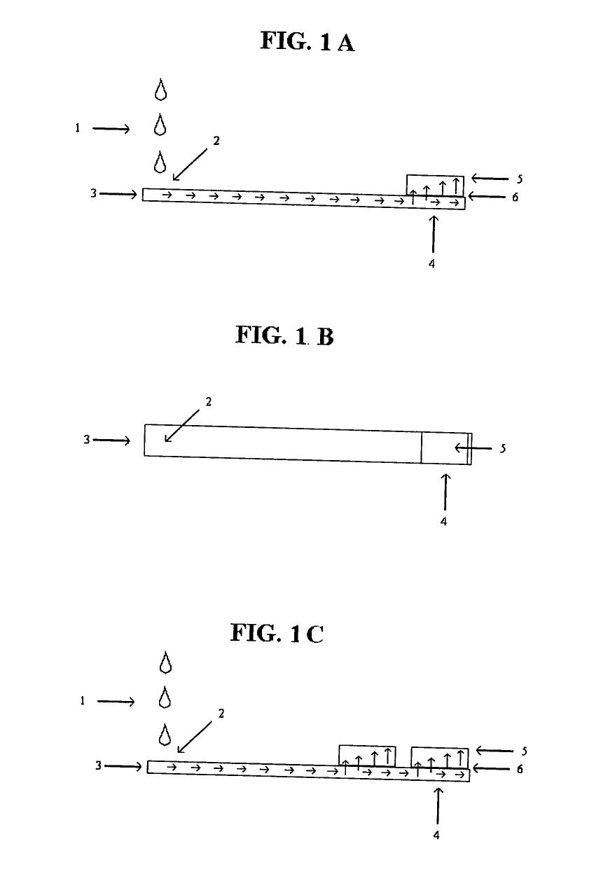

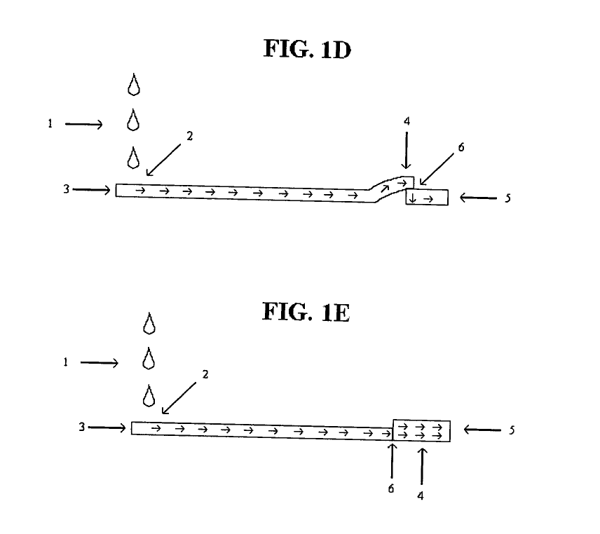

Referring now to the drawings and in particular FIGS. 1, 1A-1E, 2, and 3, there is shown an collection and assaying device which includes a container 10, in accordance with the present invention. The device generally includes an opening 17 which provides a means for collecting the biological fluid and a chamber 32 which provides a means for storing the collected fluid.

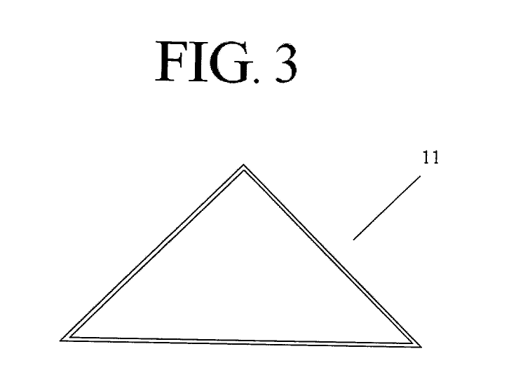

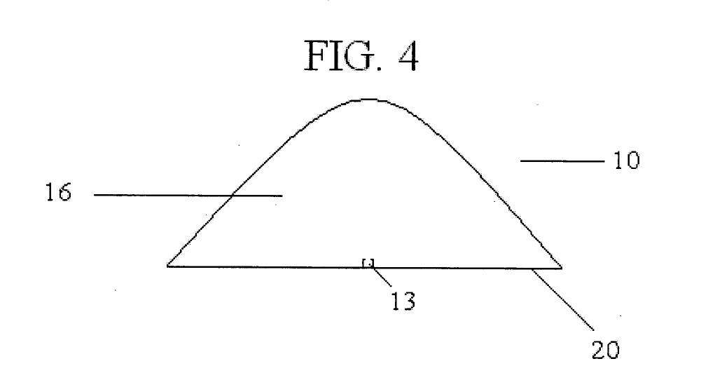

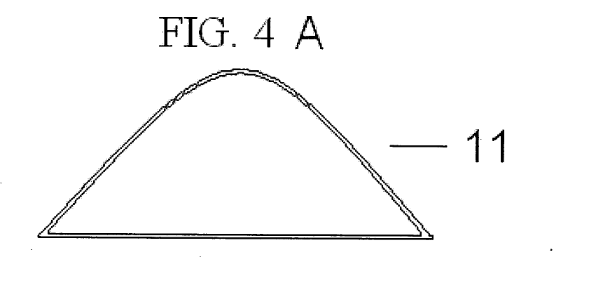

The container 10 and assaying means as illustrated by FIG. 's 1A-1E may be formed, or molded, from any suitable material such as plastic, polymers, etc., and may include a snap on lid 11 as illustrated in FIG. 3 at the top of the container 17 formed into the top 17 of the side walls 20 of the container and would be sized for accepting the lid 11. The lid 11 when snapped onto the container 10 opening 16. For typical biological collection to include urinalysis (UA), drug screening, clinical chemistry, pregnancy testing, etc., the typical container 10 capacity of about 100 to 150 mLs of liquid to accommodate split specimen requirement and additional testing.

The assaying means which may contain a plurality of separated thin layer chromatograph strips with each strip comprising means for chemically analyzing the biological fluid for a different analyte, or chromatograph, or thin layer chromatography and dry chemistry hybrid, or dry chemistry test pads attached to lateral flow device or other material assay means, as once such possible means is illustrated by FIGS. 1A-1E which can be inserted into the slots 13 of the Delta Cup.

The lateral flow device(s) hybrid (LFDH) that can be used in the Delta Cup takes the form of dry chemistry test pads that make up lateral flow hybrid devices that can be inserted into the Delta Cup slots 13. The hybrid is composed of some or all of the following compounds: test pad (usually filter paper) impregnated with buffers, and reaction components that can include indicators, surfactants or other ingredients needed for the test pad to be reactive to a specific target analyte of interest, hereinafter referred to as the test pad. The lateral flow material can be composed of any form of absorbent, solid phase carrier that is capable of transporting a fluid and in some cases can be used as a support material. The LFDH in its simplest terms is a dry chemistry test pad chemically impregnated identically to the current art for dipsticks. The test pad is then placed in (direct) contact with lateral flow paper (such as nitrocellulose) or other suitable wicking material. This device is then exposed to a fluid (urine for example). The urine (or other fluid) then migrates to the location of the test pad, saturates the test pad, and the reaction takes place. In the case of the Delta Cup the devices are exposed to fluid from the bottom of the cup. Therefore the direction of the drops and arrows for illustrative purposes are from the bottom of the cup 18 towards the top 17 of the Delta Cup.

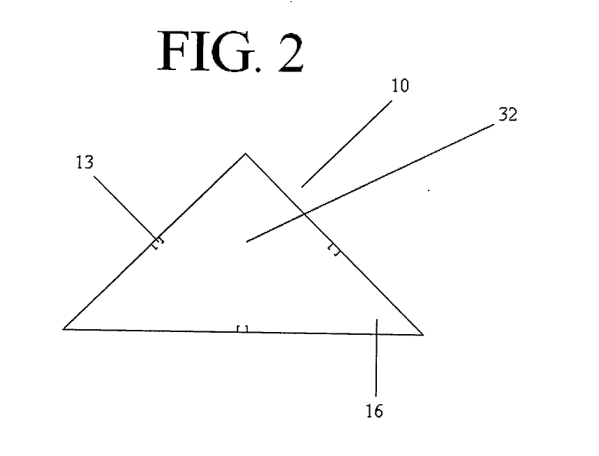

Referring now to FIG. 1A of the drawing, the liquid sample 1 is introduced from the bottom 18 of the Delta Cup illustrated as drops exposing in some manner to the sample introduction area 2 of the lateral flow material 3. The sample 1 then migrates (as illustrated by the arrows) from the sample introduction area 2 to opposite end of the lateral flow material 4 to the top of the cup 17. While the sample 1 is flowing from the sample introduction area 2 to the opposite end 4 of the lateral flow material 3 the chemically impregnated dipstick test pad 5 (which is in direct (fluid) contact 6 with the lateral flow material 3) will become saturated (acting as a wick) with the sample 1. The chemical reaction will occur between the test pad 5 and the sample 1 producing a detectable response. FIG. 1B-E all function in relatively the same manner as FIG. 1A. The only functional difference in these illustrations from the device of FIG. 1A is that the lateral flow material 4 is placed onto the top edge of the chemically impregnated dipstick test pad 5 as shown in FIG. 1D or in fluid contact such as illustrated in FIG. 1E. Thus, when the fluid sample 1 reaches the edge of the dipstick test pad 5, the test pad 5 becomes saturated with the sample 1 in the same manner as FIG. 1A and the chemical reaction takes place and a detectable response occurs. FIG. 1E again, also functions in the same manner as FIG. 1A-1D. The only functional difference in this device from that of FIG. 1A and FIG. 1E is that the lateral flow material 4 is placed next to the chemically impregnated dipstick test pad 5 (but, still in direct (fluid) contact 6 with the lateral flow material 3). All of the FIG. 's as shown function in the same novel and inventive manner. For instance, as shown in FIG. 1C multiple test pads 5 are in direct contact 6 with the lateral material 4. As the fluid migrates from one pad to the next, no cross over from one test pad 5 to the next occurs, thus, preventing cross contamination. This has never been available, taught or eluded to in the prior art. This method also allows for a specific and constant amount of fluid to reach each pad, enhancing precision, accuracy, and specificity. As shown in detail in FIGS. 1 and 2 the slots 13 for inserting the assay means can be six to ten or more per side or there can simply be one slot 13. It can be readily understood from the illustrations of the device that photocopying the device or reading the device using an instrument is made simple by the inherent design advantage of the present device. FIG. 3 illustrates the triangular shaped lid 11 that can be placed on the Delta Cup but is not required.

This detailed description as provided allows for a marked advance in the art of specimen collection, analysis and recording of results by photocopying. The present invention provides a method of specimen collection and analysis as defined above, and the method being characterized by the following steps:
a) collecting the specimen in the cup;
b) placing the lid on the cup and closing;
c) reading the result by direct observation, recording the results by photocopying the side(s) of the cup, or reading the results using an instrument.

The simplicity and novelty of the invention is unmatched in the art. This device could be easily automated and include a magnifying plastic lens that would increase visibility of the assay means results. An automation example would be to have an instrument reads the side of the container automatically and download the result to a computer. This invention is going to save the clinical diagnostic, drug of abuse testing, and other industries millions of dollars in analysis time, safety prevention and accident control, time (labor), and cost through the novel simplicity of the present invention.

To further explain the assaying device for collecting a fluid specimen and analyzing a portion of the sample, said device comprises a container means, having an opening, for collecting a specimen, and a chamber with flat side(s) (e.g. that is to say that the cup has at least one flat side), for storing said specimen. A cap means for sealing the container means opening and assay means, integrated into the said container means, for chemically analyzing said specimen, said assay means being positioned in the outside wall of the container means for enabling direct visual observation or photocopying thereof of the assay results. This device does not require the use of a plunger, tilting, pumping or other means. In other words the following is not required of the present art and can be excluded in the claims if necessary for instance the present art does not require the inflation, insertion, and closure of the device or require multiple steps as required by the prior art and the prior art are not simple efficient methods to collect and analyzed urine without the risk or contamination, spillage, or other problems. All of the prior art requires tedious and complex methods for use. For instance, the one prior requires that certain the cup be tilted prior to analysis increasing the risk of leakage and contamination as the specimen leaks out of the container. Another device requires the use of a plunger (syringe) for use and yet another requires the use of tilting and a plenum. These are just some, not all, of the limitations of the prior art.

The present device is designed for the analysis of biological specimens on site. That is to say the device can be used for the collection and analysis of the specimen within the container without removal of the specimen and without have to adjust the lid of the container, use a plunger, a plenum, or other multiple steps as required by the prior art. The specimen can be analyzed immediately at the point of collection or sent to the lab and tested the next day. The device can be used for long storage of a specimen before testing and/or for immediate analysis. This removes the risk of contamination, mislabeling, chain of custody, and cross over contamination and offers the added ability to copy results from any side of the collection device for recording of test results or the device can be easily read by an instrument providing a means of removing subjective analysis that is inherent the novel and inventive design of the present device. The assaying device of the present are wherein said device comprises a lateral flow means the allows fluid contact between the assay means and liquid introduced into the device. In addition the assaying device incorporates a means (e.g. such as a slot) that is integrated into the outside wall of the assay device that allows the assay means to be inserted into during manufacture of the device. Therefore this device is for collecting and analyzing a fluid specimen, assaying a portion of the fluid specimen comprising; containing means for collecting the said specimen; placing said specimen into said containing means; placing cap means for sealing onto the said container means; observing the assay means by direct observation, photocopying or analysis by instrumentation.

The invention has been described in detail with particular reference to a preferred embodiment and the operation thereof and it is understood that variations, modifications, and substitution of equivalent means can be effected and still remain within the spirit and scope of the invention. And all such modifications and variations are to be included within the scope of the invention as defined in the appended claims.

1. An assaying device for collecting a fluid specimen and analyzing a portion of the sample, said device comprising:
a) container means, having an opening, for collecting a specimen, and a chamber with a least one flat side, for storing said specimen;
b) cap means for sealing the container means opening;
c) assay means, integrated into the said container means, for chemically analyzing said specimen, said assay means being positioned in the outside wall of the container means for enabling direct visual observation or photocopying thereof of the assay results.

2. The assaying device according to claim 1 wherein said assay means comprises lateral flow means the allows fluid contact between the assay means and liquid introduced into the device.

3. The assaying device according to claim 1 wherein said assay means is integrated into the outside wall of the assay device.

4. A device for collecting and analyzing a fluid specimen, assaying a portion of the fluid specimen comprising;
a) containing means for collecting the said specimen;
b) placing said specimen into said containing means;
c) placing cap means for sealing onto the said container means;
d) observing the assay means by direct observation, photocopying or analysis by instrumentation.
